(19) United States
(12) Patent Application Publication
(10) Pub. No.: US 2025/0339081 A1
(43) Pub. Date: Nov. 6, 2025

(54) METHOD AND APPARATUS FOR EVALUATING AND REDUCING NEURODEVELOPMENTAL DISORDER SYMPTOM

(71) Applicant: Kayleen Hyeonji CHO, Chaska, MN (US)

(72) Inventor: Kayleen Hyeonji CHO, Chaska, MN (US)

(21) Appl. No.: 18/653,889

(22) Filed: May 2, 2024

(51) Int. Cl. A61B 5/38; A61B 5/0205; A61B 5/11; G16H 50/50

(52) U.S. Cl. CPC A61B 5/38; A61B 5/0205; A61B 5/11; G16H 50/50

(57) ABSTRACT

Provided is an apparatus for evaluating and reducing neurodevelopmental disorder symptom using a bio-signal, comprising a processor; and a memory comprising one or more sequences of instructions which, when executed by the processor, causes steps to be performed comprising; receiving a user's bio-signal through a biosensor, evaluating the user's neurodevelopmental disorder symptom by extracting feature information from the bio-signal based on a machine learning model, and selecting at least one of a plurality of music that matches the neurodevelopmental disorder symptom and transmitting the music to the user to reduce the neurodevelopmental disorder symptom.

### TECHNICAL FIELD

The present disclosure relates to evaluating neurodevelopmental disorder symptom, more particularly, to an apparatus and method for evaluating neurodevelopmental disorder such as alexithymia and autism using a bio-signal and for reducing a neurodevelopmental disorder symptom.

### DESCRIPTION OF THE RELATED ART

In the ever-evolving landscape of healthcare and neurodevelopmental disorders, the demand for innovative methods and technologies to assess and ameliorate symptoms continues to grow. Neurodevelopmental disorders pose unique challenges to individuals, families, and communities worldwide, impacting cognitive, emotional, and social functioning. Conditions such as Autism Spectrum Disorder (ASD), Attention-Deficit/Hyperactivity Disorder (ADHD), and others are characterized by atypical patterns of neural development that manifest in a variety of behavioral and cognitive symptoms. While traditional therapeutic interventions have been instrumental in addressing these challenges, there is a growing interest in alternative and complementary approaches that tap into the power of music therapy.

### SUMMARY OF THE DISCLOSURE

In one aspect of the present disclosure, an apparatus for evaluating and reducing neurodevelopmental disorder symptom, comprises a biosensor for receiving a bio-signal from an user, a signal analyzer for evaluating the user's neurodevelopmental disorder symptoms by analyzing the bio-signal based on a reference signal data, and a controller for selecting at least one of a plurality of music to reduce the neurodevelopmental disorder symptoms and transmitting the music the user.

In another aspect of the present disclosure, an apparatus for generating a biometric image, comprises a processor; and a memory comprises one or more sequences of instructions which, when executed by the processor, causes steps to be performed comprising: receiving a user's bio-signal through a biosensor, evaluating the user's neurodevelopmental disorder symptom by extracting feature information from the bio-signal based on a machine learning model, and selecting at least one of a plurality of music that matches the neurodevelopmental disorder symptom and transmitting the music to the user to reduce the neurodevelopmental disorder symptom.

Desirably, the bio-signal may include at least one of heart rate, heart rate variability, respiratory rate, temperature, and skin conductance.

Desirably, the biosensor may include at least one of a photoplethysmography (PPG) sensor, an electrodermal activity (EDA) sensor, and a temperature sensor.

Desirably, the machine learning model may additionally extract the feature information from a user's clinical data.

Desirably, the biosensor may include a motion sensor to measure the user's movement.

In further aspect of the present disclosure, a method for evaluating and reducing neurodevelopmental disorder symptom, comprises receiving a user's bio-signal through a biosensor, evaluating the user's neurodevelopmental disorder symptom from the user's bio-signal, and selecting at least one of a plurality of music that matches the neurodevelopmental disorder symptom and transmitting the music to the user to reduce the neurodevelopmental disorder symptom.

Desirably, the user's neurodevelopmental disorder symptoms may be evaluated by analyzing the user's bio-signal based on a reference signal data.

Desirably, the user's neurodevelopmental disorder symptoms may be evaluated by extracting feature information from the user's bio-signal based on a machine learning model.

### BRIEF DESCRIPTION OF THE DRAWINGS

References will be made to embodiments of the disclosure, examples of which may be illustrated in the accompanying figures. These figures are intended to be illustrative, and not limiting. Although the disclosure is generally described in the context of these embodiments, it should be understood that it is not intended to limit the scope of the disclosure to these particular embodiments.

FIG. 1 is an exemplary block diagram of an apparatus for evaluating neurodevelopmental disorder according to a first embodiment of the present disclosure.

FIG. 2 is a block diagram illustrating an analysis method of a signal analyzer for a bio-signal according to the first embodiments of the present disclosure.

FIG. 3 is an exemplary block diagram of an apparatus for evaluating neurodevelopmental disorder according to a second embodiment of the present disclosure.

FIG. 4 is a block diagram illustrating an analysis method of a signal analyzer for a bio-signal according to the second embodiments of the present disclosure.

FIG. 5 is an exemplary block diagram of an apparatus for evaluating neurodevelopmental disorder according to a third embodiment of the present disclosure.

FIG. 6 is a block diagram illustrating an analysis method of a signal analyzer for a bio-signal according to the third embodiments of the present disclosure.

FIG. 7 is an exemplary block diagram of an apparatus for evaluating and reducing neurodevelopmental disorder according to a first embodiment of the present disclosure.

FIG. 8 is a schematic diagram for explaining the operation of an apparatus according to embodiments of the present disclosure.

FIG. 9 is a schematic diagram of an illustrative apparatus for evaluating and reducing neurodevelopmental disorder symptoms according to a second embodiment of the present disclosure.

FIG. 10 is a schematic diagram of an illustrative system for evaluating and reducing neurodevelopmental disorder symptoms according to embodiments of the present disclosure.

FIG. 11 is an exemplary diagram illustrating a process for extracting feature information of bio-signal data by a processor according to embodiments of the present disclosure.

FIG. 12 is an exemplary flowchart showing a first method for evaluating and reducing neurodevelopmental disorder symptoms by an apparatus according to embodiments of the present disclosure.

FIG. 13 is an exemplary flowchart showing a second method for evaluating and reducing neurodevelopmental disorder symptoms by an apparatus according to embodiments of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity of referring to performing machine learning by a processing module such as a processor, a CPU, an application processor, micro-controller, and so on.

An “feature(s)” is defined as a group of one or more descriptive characteristics of subjects that can discriminate for a neurodevelopmental disorder symptom. The feature can be a numeric attribute.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

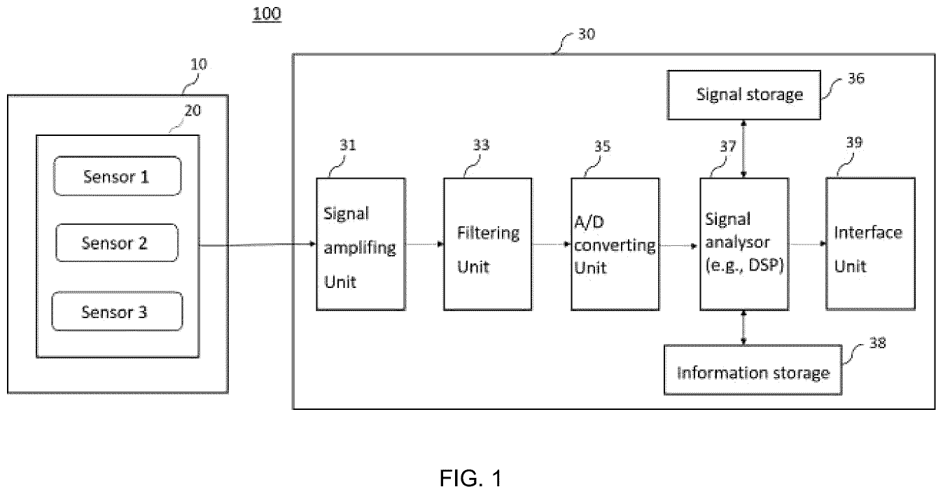

FIG. 1 is an exemplary block diagram of an apparatus for evaluating neurodevelopmental disorder according to a first embodiment of the present disclosure.

As depicted, the apparatus 100 may include a body 10, a biosensor 20 and a controller 30. The body 10 may be configured in the form of a headphone or earphone that can support a biosensor by contacting a user's specific part such as the external auditory canal or the earlobe area. The body 10 may also have various shapes that can efficiently support the biosensor according to a body part of the user (user).

The biosensor 20 may contact the body part of the user and detect blood flow rate, blood pressure, blood flow change, etc. using various bio-signals (e.g., pulse) detected from a specific part of the user's body. The biosensor 200 may also measure heart rate, heart rate variability and respiratory rate using the blood volume changes. The biosensor 20 may include one or more sensors using light, electrical conductance, or pressure, and may include a photoplethysmography (PPG) sensor, an electrodermal activity (EDA) sensor, and a temperature sensor. For example, the PPG sensor may use a light source and a photodetector at the surface of the skin to measure the volumetric variations of blood circulation. The PPG sensor may be used in reflectance mode in which the photodetector is positioned along the light source on the same side to measure the reflected light from the skin. The EDA sensor may measure the electrical properties of the skin which change. These changes are caused by alterations in sweat secretion and sweat gland activity as a result of changing sympathetic nervous system activity.

The controller 30 may be a processing module that is electrically and mechanically coupled to the body 10 and the biosensor 20, and automatically processes electrical signals. In embodiments, the controller 30 may be a CPU, AP (Application Processor), microcontroller, etc., but is not limited thereto. In addition, the controller 30 may communicate with the body 10 and external devices wired or wirelessly and perform signal processing on electrical signals detected from the biosensor 20. In embodiments, the controller 30 may include a signal amplifying unit 31, a filtering unit 33, an analog/digital (A/D) converting unit 35, a signal storage 36, a signal analyzer 37, an information storage 38, and a communication interface unit 39.

In embodiments, the signal amplifying unit 31 may amplify the bio-signals detected by the biosensor 20 using an instrumental amplifier. In embodiments, the filtering unit 33 may filter out unnecessary components, such as noise components, from the bio-signals amplified by the signal amplifying unit 31. The filtered bio-signal may be converted to a digital signal by the analog/digital converting unit 35 to be converted to a digital bio-signal. In embodiments, the signal analyzer 37 may be a digital imaging processor that analyzes digital bio-signals to diagnose neurodevelopmental disorder such as alexithymia and autism. The specific analysis method will be described later. In embodiments, the signal storage 36 may store the digital bio-signals analyzed and extracted by the signal analyzer 37. Reference information for bio-signals belonging to the normal or abnormal group in medical diseases may be preset and stored in the signal storage 36. In embodiments, the information storage 38 may input the user's physical information using an input device. For example, the physical information may be user's age, gender, height, weight, and so on. In addition, medical standard bio-signal data according to the user's physical information may be stored into the information storage 38. In embodiments, the interface unit 39 may be a wired or wireless communication interface that can transmit the analysis results of digital bio-signals analyzed by the signal analyzer 37 to an external device.

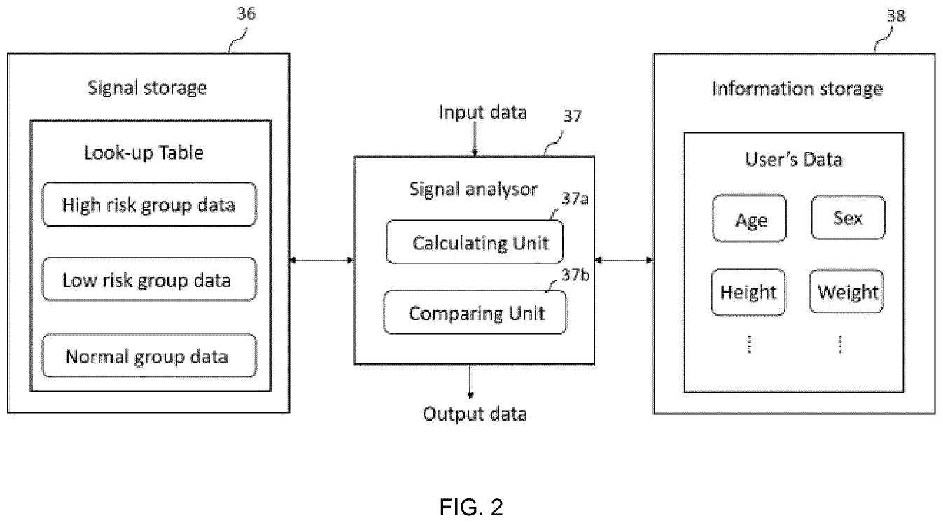

FIG. 2 is a block diagram illustrating an analysis method of a signal analyzer for a bio-signal according to the first embodiments of the present disclosure.

As depicted, the signal analyzer 37 may include a calculating unit 37a and a comparing unit 37b. When a bio-signal of a user is input to the signal analyzer 37, the internal calculating unit 37a may process the bio-signal to convert it into digital numerical values, and the comparing unit 37b may compare the digital bio-signal numerical values with pre-set threshold values to output signal data divided into normal and abnormal groups based on whether there is a difference beyond a certain range. In embodiments, when the digital bio-signal numerical values represent by a waveform of the bio-signal, the values may be peak values of the waveform of the bio-signal. In this case, the signal analyzer 37 may output the signal data divided into normal and abnormal groups by matching and utilizing the user's clinical information stored in an information storage 38.

In embodiments, if the measured digital bio-signal numerical values indicate an abnormal group, the signal analyzer 37 may divide and output the degree of risk by comparing with high-risk group data and low-risk group data included in a look-up table of a signal storage 36. The abnormal group signifies being in conditions such as alexithymia, ASD and ADHD.

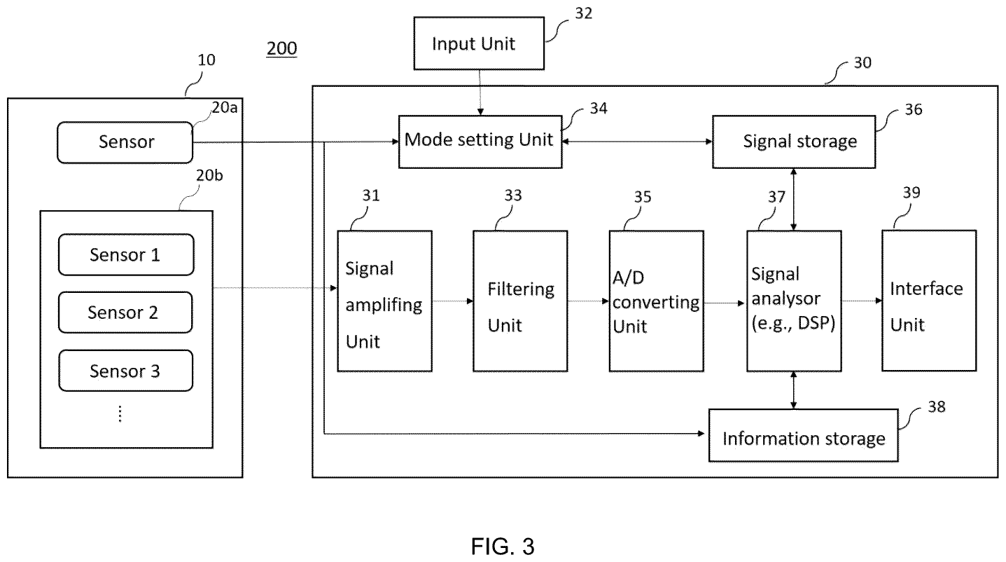

FIG. 3 is an exemplary block diagram of an apparatus for evaluating neurodevelopmental disorder according to a second embodiment of the present disclosure.

As depicted, the apparatus 200 may include a body 10, a biosensor 20a, 20b, a controller 30, and an input unit 32. The components included in the body 10 and the controller 30 are similar to those described in FIG. 1, and therefore, the description of their operation and functions is omitted here. However, the control unit 30 according to this embodiment may additionally include a mode setting unit 34, and the input unit 32 for setting mode may be integrated into the body 10.

The biosensor 20a, 20b may include a first sensor 20a and a second sensor 20b. The first sensor 20a may be integrated into the body 10 or physically separated from it and adhered to a designated area of the user's body in patch form. As an example, the first sensor 20a may include gyro sensors or acceleration sensors capable of measuring the user's movements. The data on movement measured by the first sensor 20a may be stored in an information storage 38 and become one of the parameters for evaluating neurodevelopmental disorder during bio-signal analysis. The second sensor 20b is similar to the corresponding component 20 described in FIG. 1, and thus, the description of its operation and functions is also omitted.

Meanwhile, the setting mode may be stored as a stabilized mode or a non-stabilized mode and may be set to various modes depending on other environmental factors of the user. For instance, the stabilized mode may be activated when the user is sleeping or maintaining a posture with minimal movement, while the non-stabilized mode may be activated when the user is engaging in activities with significant movement. The setting mode may be automatically adjusted to the stabilized mode, or the non-stabilized mode based on the values measured by the first sensor 20a (i.e., motion sensor) under the control of the controller 30, or it may be manually adjusted to various modes by the user via the input unit 32.

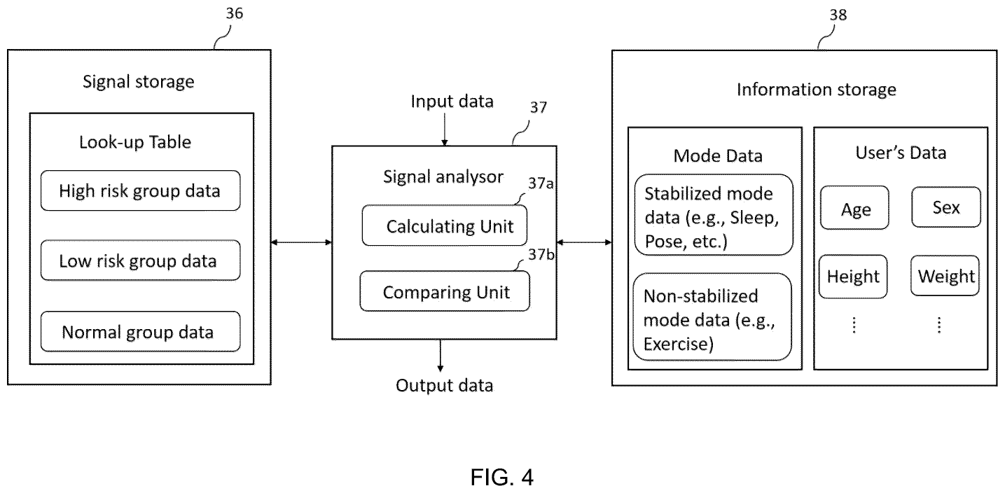

FIG. 4 is a block diagram illustrating an analysis method of a signal analyzer for a bio-signal according to the second embodiments of the present disclosure.

As depicted, the signal analyzer 37 is similar to components corresponding to those described in FIG. 2, and therefore, the description of its operation and functionality is omitted. However, the signal analyzer 37 may analyze the user's bio signals based on additional setting mode data obtained through the first sensor 20a or directly inputted by the user, enabling the evaluation of neurodevelopmental disorder such as alexithymia and autism.

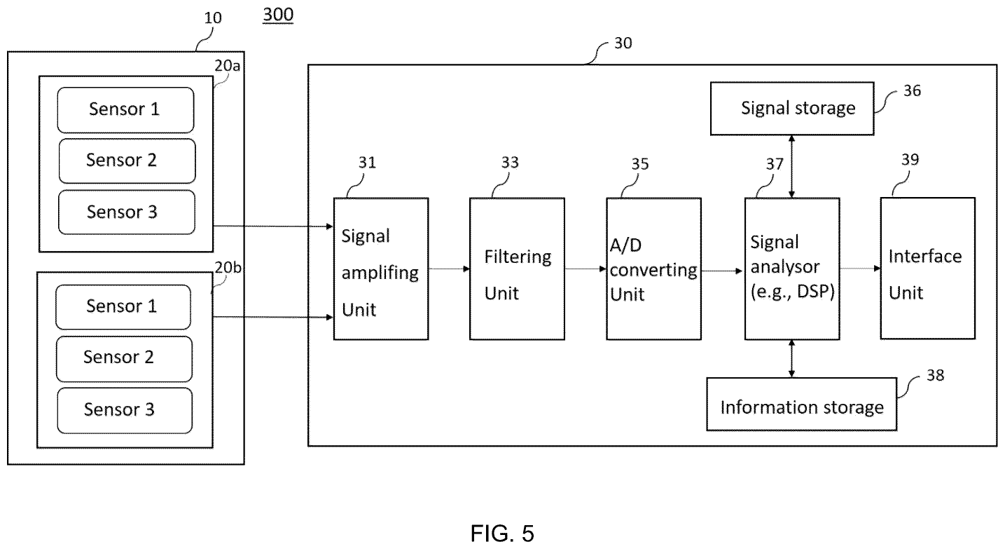

FIG. 5 is an exemplary block diagram of an apparatus for evaluating neurodevelopmental disorder according to a third embodiment of the present disclosure.

As depicted, the apparatus 300 is similar to components corresponding to those described in FIG. 1, and therefore, the description of their operation and functionality is be omitted. However, the biosensor includes a first sensor 20a and a second sensor 20b, which are similar to the components 20 described in FIG. 1. For example, the first and second sensors may be attached to the left and right blood vessels of the subject (e.g., earlobe area) to measure bio-signals, and the signal analyzer 37 can diagnose the presence of status by analyzing the measured bio-signals from left and right blood vessels. Each of the first sensor 20a and the second sensor 20b may include at least one of the photoplethysmography (PPG) sensor, an electrodermal activity (EDA) sensor, and a temperature sensor.

Two sensors 20a, 20b on both sides may help determine a more accurate reading by finding readings from both sides; the detected signals should be relatively homogeneous and be accurate with any readings that result in hospital or formal setting ECG readings. If potential differences in readings are shown over long periods, the apparatus 300 could potentially be able to detect any health issues that the user may be experiencing where the blood flow is not similar when traveling on the left side of the head and the right. This could potentially help diagnose or find issues that the user may be suffering with such as aneurysms, atherosclerosis, venous disease, heart attack, and more.

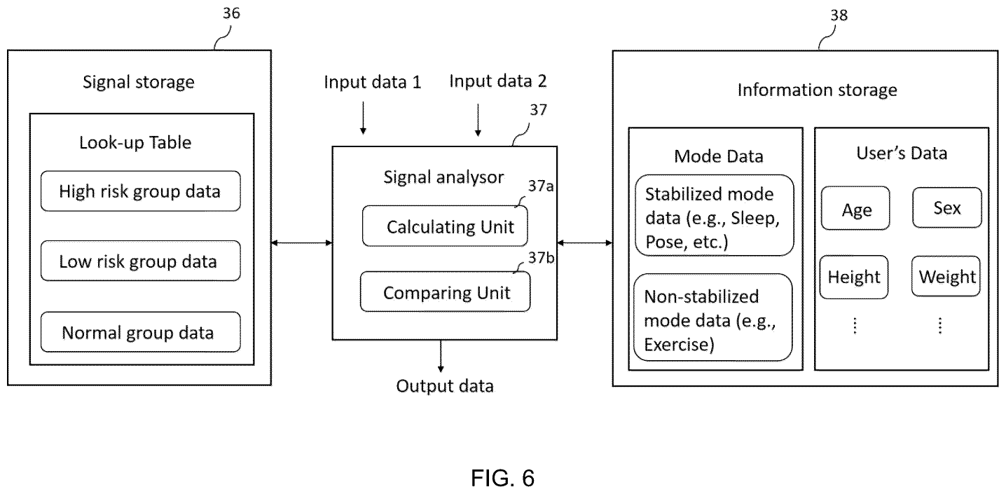

FIG. 6 is a block diagram illustrating an analysis method of a signal analyzer for a bio-signal according to the third embodiments of the present disclosure.

As depicted, the signal analyzer 37 is similar to components corresponding to those described in FIG. 2, and therefore, the description of its operation and functionality is omitted. However, the signal analyzer 37 according to this embodiment may compare and analyze the first and second bio-signals measured by the first sensor 20a and the second sensor 20b. For example, when the first and second bio-signals are outputted as waveforms, the signal analyzer 37 may compare the peak values of the waveforms to calculate the difference between the first and second bio-signals and may subsequently compare the difference value with a threshold value within the normal range to diagnose the presence of neurodevelopmental disorder.

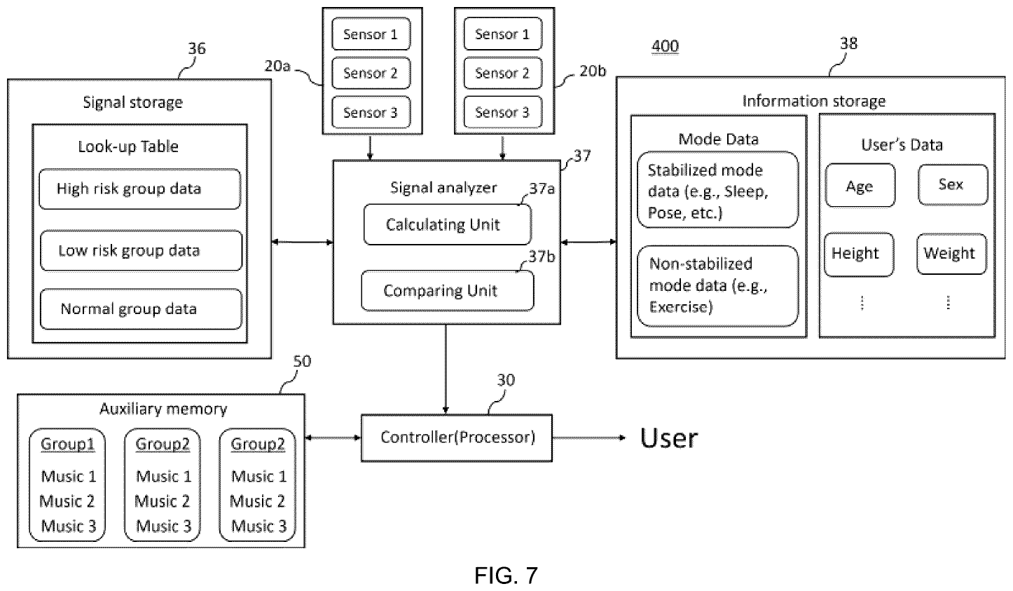

FIG. 7 is an exemplary block diagram of an apparatus for evaluating and reducing neurodevelopmental disorder symptom according to a first embodiment of the present disclosure.

As depicted, the signal analyzer 37 may analyze a first bio-signal and a second bio-signal measured by a first sensor 20a and a second sensor 20b, respectively. In embodiments, each of the first sensor 20a and the second sensor 20b may include at least one of a PPG sensor, an EDA sensor, and a temperature sensor. The signal analyzer 37 may detect a biometric signal such as heart rate, skin conductance, temperature through the sensors 20a, 20b and may calculate the biometric signal by quantifying them numerically. In embodiments, in the signal storage, the pre-stored risk group data according to the level of bio-signals may be stored in a form of a look-up table.

if the measured digital bio-signal numerical values indicate an abnormal group, the signal analyzer 37 may divide and output the degree of risk by comparing with high-risk group data and low-risk group data included in the look-up table of a signal storage 36. The abnormal group signifies being in conditions (i.e., neurodevelopmental disorder symptom) such as alexithymia, ASD and ADHD. At this time, the signal analyzer 37 may additionally output the degree of the neurodevelopmental disorder symptom based on a mode data or user's data storage in the information storage 38. In embodiments, the controller 30 may select a music group that matches the output level of risk and deliver it to the user as a sound to reduce neurodevelopmental disorder symptoms.

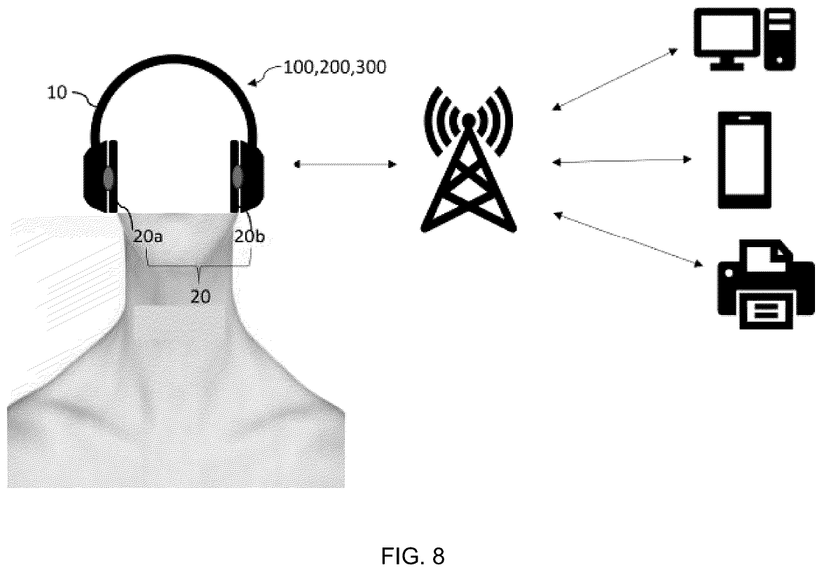

FIG. 8 is a schematic diagram for explaining the operation of an apparatus according to embodiments of the present disclosure.

As depicted, the apparatus 100, 200, 300 may transmit the analysis results of bio-signals to an external device, such as the user's PC, laptop, PDA, or mobile terminal. This can allow the user to periodically measure their health status and perform diagnosis by an external expert doctor or a specialized analysis program in the ubiquitous medical environment.

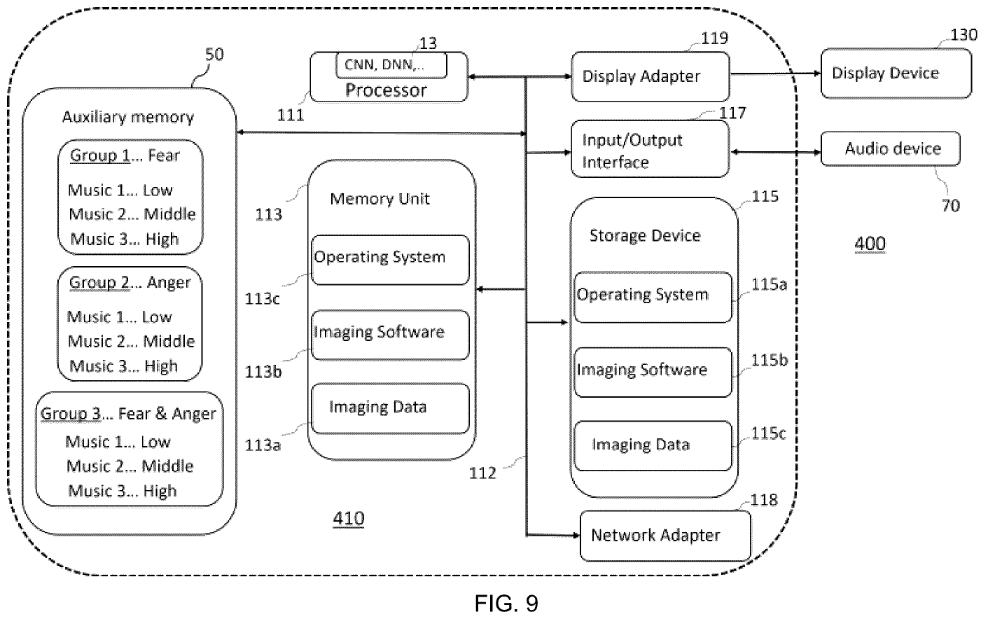

FIG. 9 is a schematic diagram of an illustrative apparatus for evaluating and reducing neurodevelopmental disorder symptoms according to a second embodiment of the present disclosure.

As depicted, the apparatus 400 may include an audio device 70, a computing device 410, a display device 130. In embodiments, the computing device 410 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 400 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems. In embodiments, the audio device 70 may include a body and at least biosensor installed on the body. The bio-signal acquired by the audio device 70 may include bio-signal information such as heart rate, heart rate variability, respiratory rate, temperature, and electrical properties of the skin. The bio-signal may be stored in the memory unit 113 or the storage device 115 in time series or may be provided to the processor 111 through the input/output interface 117 and processed based on a machine learning model 13.

In embodiments, the processor 111 is, but is not limited to, a processing module, a Computer Processing Unit (CPU), an Application Processor (AP), a microcontroller, and/or a digital signal processor. In addition, the processor 111 may communicate with a hardware controller such as the display adapter 119 to display a user interface on the display device 130. In embodiments, the processor 111 may access the memory unit 113 and execute commands stored in the memory unit 113 or one or more sequences of instructions to control the operation of the apparatus 400. The commands or sequences of instructions may be read in the memory unit 113 from computer-readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

In embodiments, the apparatus 400 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing device.

In embodiments, the network may be, but is not limited to, more than one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 410 may include a variety of computer-readable medium or media. The computer-readable medium or media may be any available medium or media that is accessible by the computing device 410. For example, the computer-readable medium or media may include, but is not limited to, both volatile and non-volatile media, removable or non-removable media.

In embodiments, the memory unit 113 may typically stores a database of bio-signals that are used by the machine learning model 13. Although the database could be at some other location that is external to the remote computing devices 500, 600, 700 and accessible by the processor 111 via a network. the memory unit 113 may store a driver, an application program, data, and a database for operating the apparatus 400 therein. In addition, the memory unit 113 may include a computer-readable medium in a form of a volatile memory such as a random-access memory (RAM), a non-volatile memory such as a read only memory (ROM), and a flash memory. For instance, it may be, but is not limited to, a hard disk drive, a solid-state drive, and/or an optical disk drive.

In embodiments, each of the memory unit 113 and the storage device 115 may be program modules such as the imaging software 113b, 115b and the operating systems 113c, 115c that can be immediately accessed so that a data such as the imaging data 70a, 70b is operated by the processor 111.

In embodiments, the machine learning model 13 may be trained to classify neurodevelopmental features contained in bio-signals and to evaluate or predict, based on the classification, the presence of neurodevelopmental disorder symptoms in a human subject. The manner in which training may be performed and the manner in which the apparatus 100 is used to predict or evaluate the presence of the neurodevelopmental disorder symptoms in a human subject are described below. Once trained, the machine learning model 13 may analyze bio-signals detected by a bio-signals sensor installed into the audio device 70 to identify and classify feature information contained in the bio-signals. Based on the feature information, the machine learning model 13 may predict or evaluate the presence of neurodevelopmental disorder symptoms such as autism spectrum disorder (ASD) and attention-deficit/hyperactivity disorder (ADHD) in human subject. In embodiments, Symptoms of neurodevelopmental disorders may appear as fear or anger, and the degree of these symptoms can be expressed numerically. Depending on the symptom and its severity, the processor 1111 may select a pre-stored music group and transmit it to the user as a sound to reduce the symptom or prevent the symptom from occurring. In embodiments, the music groups may be stored into an auxiliary memory 50 of the computing device 410 and the music may be transmitted to the user through the audio device 70. In embodiments, the machine learning model 13 may be installed into at least one of the processor 111, the memory unit 113 and the storage device 115. The machine learning model 13 may use, but is not limited to, at least one of a deep neural network (DNN), a convolutional neural network (CNN) and a recurrent neural network (RNN), which are one of the machine learning algorithms.

If the apparatus 400 includes more than one computing device 410, then the different computing devices may be coupled to each other such that images, data, information, instructions, etc. can be sent between the computing devices. For example, one computing device may be coupled to additional computing device(s) by any suitable transmission media, which may include any suitable wired and/or wireless transmission media known in the art. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

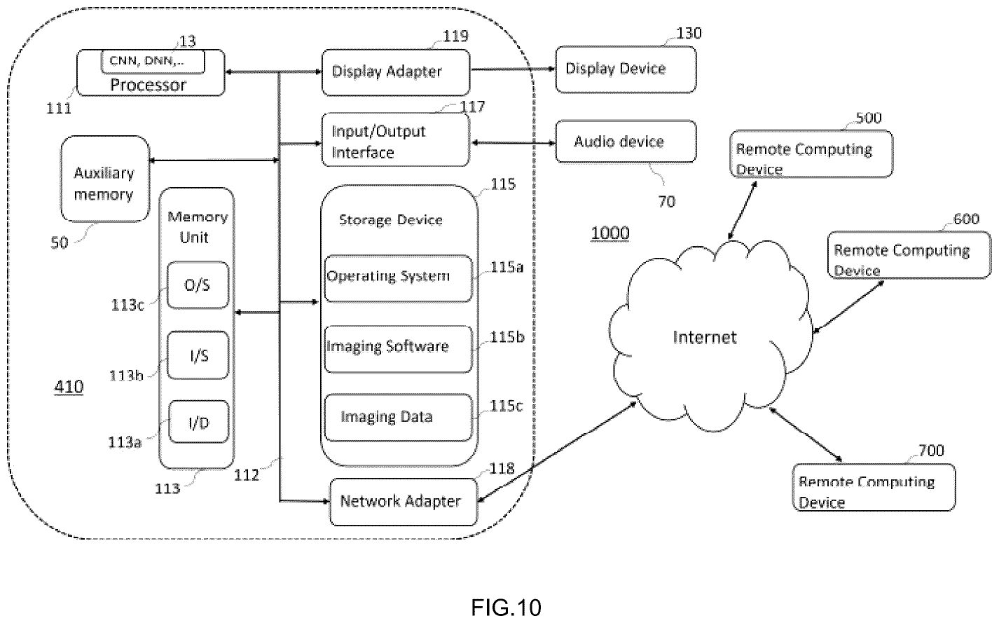

FIG. 10 is a schematic diagram of an illustrative system for evaluating and reducing neurodevelopmental disorder symptoms according to embodiments of the present disclosure.

As depicted, the system 1000 may include a computing device 410 and one and more remote computing devices 500, 600, 700. In embodiments, the computing device 410 and the remote computing devices 500, 600, 700 may be connected to each other through a network. The components of the system 1000 are similar to their counterparts in FIG. 9. In embodiments, each of remote computing devices 500, 600, 700 may be similar to the computing device 410 in FIG. 9. For instance, each of remote computing devices 500, 600, 700 may include each of the subsystems that include the processor 111, the memory unit 113, an operating system 113c, 115a, an imaging software 113b, 115b, an imaging data 113a, 115c, a network adapter 118, a storage device 115, an input/output interface 117 and a display adapter 119. Each of remote computing devices 500, 600, 700 may further include a display device 130 and an audio device 70. In embodiments, the system bus 112 may connect the subsystems to each other.

In embodiments, the computing device 410 and the remote computing devices 500, 600, 700 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present invention may also be implemented into other computing devices and systems. Furthermore, aspects of the present invention may be implemented in a wide variety of ways including software (including firmware), hardware, or combinations thereof. For example, the functions to practice various aspects of the present invention may be performed by components that are implemented in a wide variety of ways including discrete logic components, one or more application specific integrated circuits (ASICs), and/or program-controlled processors. It shall be noted that the manner in which these items are implemented is not critical to the present invention.

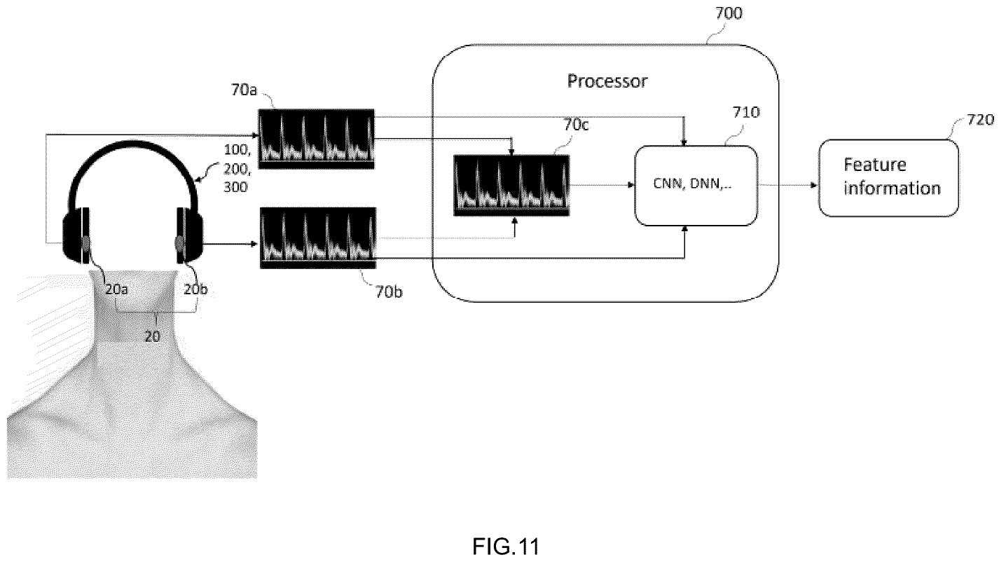

FIG. 11 is an exemplary diagram illustrating a process for extracting feature information of bio-signal data by a processor according to embodiments of the present disclosure.

As depicted, when the first bio-signal data 70a and the second bio-signal data 70b measured from the left and right ears of the subject, respectively, are input to the processor 700, the processor may generate a new third bio-signal data 70c by overlapping the first bio-signal data 70a and the second bio-signal data 70b, and the generated third bio-signal data 70c is input to a machine learning model 710. The processor 700 may extract feature information 720 from the third bio-signal data 70c based on the machine learning model 710 included therein. In alternatively, when the first bio-signal data 70a and the second bio-signal data 70b are directly input to the machine learning model 710, the processor 700 may extract feature information from the first bio-signal data 70a and the second bio-signal data 70b, respectively, based on the machine learning model 710. The processor 700 may be the processor 111 included in the computing device 410 of FIGS. 9 and 10.

The feature information 720 is information that can assist an entity such as a medical staff in predicting neurodevelopmental disorder symptoms, for example, it may be information about an abnormal signal (e.g., mismatch pulse) in which the signal (pulse) recognized within the third bio-signal data 70c does not match the signals (pulses) recognized within the first bio-signal data 70a or the second bio-signal data 70b. Alternatively, the feature information 720 may be an irregular abnormal signal (e.g., various pulses with different peaks, different periods) in the signal (pulse) itself recognized within the first bio-signal data 70a or the second bio-signal data 70b. In addition, the feature information 720 may be irregular abnormal signals (e.g., various pulses with different peaks, pulses with different periods, pulses with various inflection points) in the signal (pulse) itself recognized in the third bio-signal data 70c. The feature information 720 may be stored in the memory unit 113 or the storage device 115. Meanwhile, if the processor 700 may extract the feature information of the first bio-signal data or the second bio-signal data and the feature information of the third bio-signal data, the processor 700 may compare them and display them through a display device.

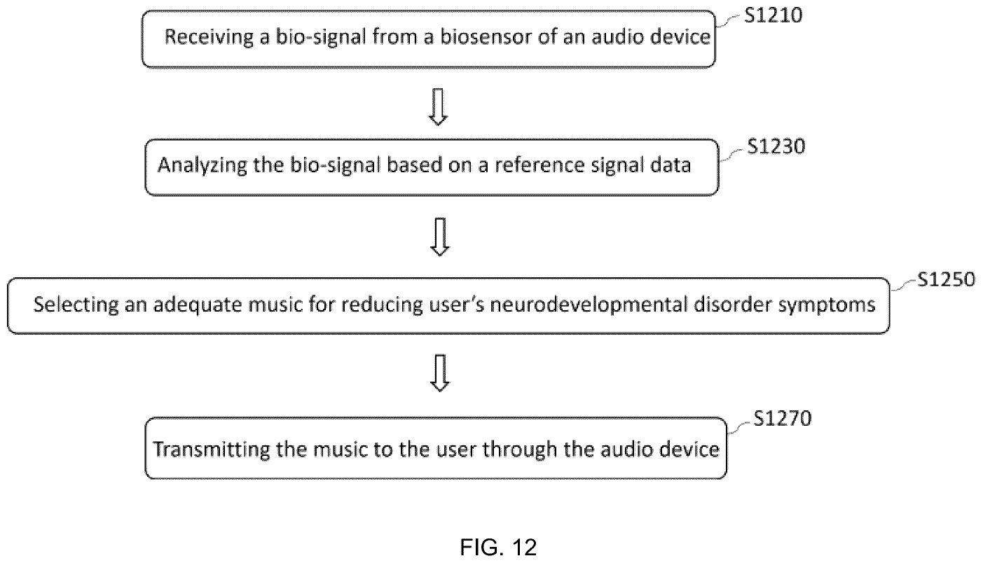

FIG. 12 is an exemplary flowchart showing a first method for evaluating and reducing neurodevelopmental disorder symptoms by an apparatus according to embodiments of the present disclosure.

Evaluating and reducing neurodevelopmental disorder symptoms may be performed by sensors, signal analyzer and controller in the apparatus 400 illustrated in FIG. 7. At step S1210, a bio-signal is received from a biosensor of an audio device. In embodiments, the bio-signal may be any of a variety of bio-signals that can measure neurodevelopmental disorder symptoms. For example, the bio-signal may include heart rate, temperature, etc. At step S1230, when the bio-signal is input into a signal analyzer, the signal analyzer may evaluate the neurodevelopmental disorder symptoms by analyzing the bio-signal on a reference signal data. In embodiments, the reference signal data may be numerical data on the neurodevelopmental disorder symptoms severity as a pre-set data in a storage. At step S1250, the controller may select a music group stored in a memory to reduce the symptoms that appear according to measured bio-signals. In embodiments, the type of music may be of various genres, mainly music with low frequencies. At step S1270, the selected music may be transmitted to the user through an audio device such as a headphone, an earphone.

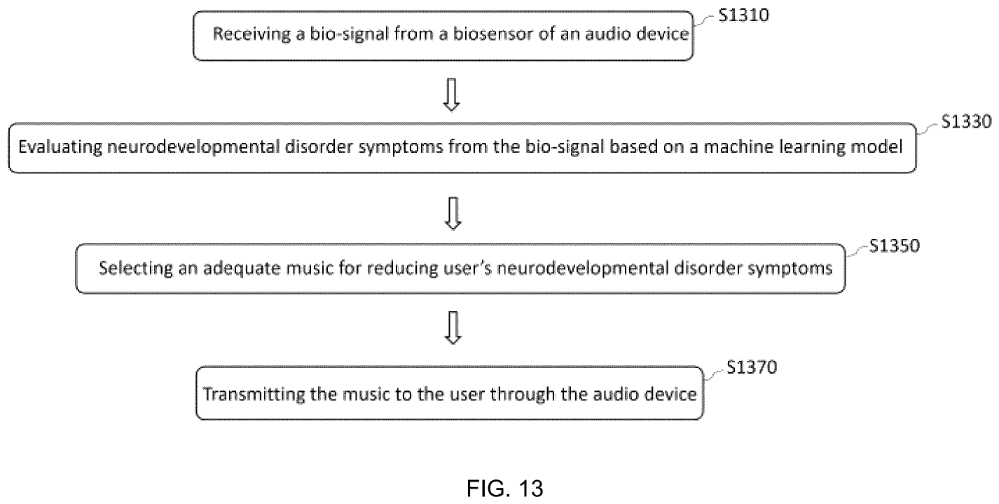

FIG. 13 is an exemplary flowchart showing a second method for evaluating and reducing neurodevelopmental disorder symptoms by an apparatus according to embodiments of the present disclosure.

Evaluating and reducing neurodevelopmental disorder symptoms may be performed by sensors, processor in the apparatus 400 illustrated in FIG. 9. At step S1310, a bio-signal is received from a biosensor of an audio device. In embodiments, the bio-signal may be any of a variety of bio-signals that can measure neurodevelopmental disorder symptoms. For example, the bio-signal may include heart rate, temperature, etc. At step S1330, from the received bio-signal, the presence of symptoms may be checked based on a machine learning model. At this time, the symptoms may be evaluated by extracting feature information from the bio-signals through various machine learning models. At step S1350, the processor may select a music group stored in a memory to reduce the symptoms that appear according to measured bio-signals. In embodiments, the type of music may be of various genres, mainly music with low frequencies. At step S1370, the selected music may be transmitted to the user through an audio device such as a headphone, an earphone.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store, or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. An apparatus for evaluating and reducing neurodevelopmental disorder symptom, comprising:
a biosensor for receiving a bio-signal from an user;
a signal analyzer for evaluating the user's neurodevelopmental disorder symptoms by analyzing the bio-signal based on a reference signal data; and
a controller for selecting at least one of a plurality of music to reduce the neurodevelopmental disorder symptoms and transmitting the music the user.

2. The apparatus of claim 1, wherein the bio-signal includes at least one of heart rate, heart rate variability, respiratory rate, temperature, and skin conductance.

3. The apparatus of claim 1, wherein the biosensor includes at least one of a photoplethysmography (PPG) sensor, an electrodermal activity (EDA) sensor, and a temperature sensor.

4. The apparatus of claim 3, wherein the signal analyzer additionally analyzes the user's clinical data and mode data.

5. An apparatus for evaluating and reducing neurodevelopmental disorder symptom, comprising:
a processor; and
a memory comprising one or more sequences of instructions which, when executed by the processor, causes steps to be performed comprising:
receiving a user's bio-signal through a biosensor evaluating the user's neurodevelopmental disorder symptom by extracting feature information from the bio-signal based on a machine learning model; and
selecting at least one of a plurality of music that matches the neurodevelopmental disorder symptom and transmitting the music to the user to reduce the neurodevelopmental disorder symptom.

6. The apparatus of claim 5, wherein the bio-signal includes at least one of heart rate, heart rate variability, respiratory rate, temperature, and skin conductance.

7. The apparatus of claim 5, wherein the biosensor includes at least one of a photoplethysmography (PPG) sensor, an electrodermal activity (EDA) sensor, and a temperature sensor.

8. The apparatus of claim 5, wherein the machine learning model additionally extracts the feature information from a user's clinical data.

9. The apparatus of claim 5, wherein the biosensor includes a motion sensor to measure the user's movement.

10. A method for evaluating and reducing neurodevelopmental disorder symptom, comprising:
receiving a user's bio-signal through a biosensor evaluating the user's neurodevelopmental disorder symptom from the user's bio-signal; and
selecting at least one of a plurality of music that matches the neurodevelopmental disorder symptom and transmitting the music to the user to reduce the neurodevelopmental disorder symptom.

11. The method of claim 10, wherein the user's neurodevelopmental disorder symptoms are evaluated by analyzing the user's bio-signal based on a reference signal data.

12. The method of claim 10, wherein the user's neurodevelopmental disorder symptoms are evaluated by extracting feature information from the user's bio-signal based on a machine learning model.
